(19) United States
(12) Patent Application Publication
(10) Pub. No.: US 2022/0193425 A1
(43) Pub. Date: Jun. 23, 2022

(54) ACCESSORY FOR TRANSPORTATION AND STORAGE OF AN AUTONOMOUS CARDIAC IMPLANT OF THE LEADLESS CAPSULE TYPE

(71) Applicant: CAIRDAC, Antony (FR)

(72) Inventors: Alaa Makdissi, Paris (FR); Willy Regnier, Longjumeau (FR); An Nguyen-Dinh, La Riche (FR)

(21) Appl. No.: 17/463,996

(22) Filed: Sep. 1, 2021

(30) Foreign Application Priority Data
Dec. 22, 2020 (EP) 20315501.5

(51) Int. Cl. A61N 1/375; A61N 1/05; A61N 1/372; A61N 1/378; H02N 2/18

(52) U.S. Cl. CPC A61N 1/3756; A61N 1/059; H02N 2/188; A61N 1/3785; H02N 2/181; A61N 1/37217

(57) ABSTRACT

The implant comprises a tubular body housing an energy harvesting module adapted to convert external stresses applied to the implant into electrical energy, by means of an inertial pendular unit comprising an elastically deformable element coupled to an inertial mass, as well as a rechargeable battery adapted to be recharged by the energy harvesting module, the battery being previously charged at an initial charge level. The accessory comprises an external source of electrical energy for the temporary storage of an electrical energy during the transportation and storage of the implant, the external source being physically separated from the implant. A temporary electrical coupling link from the external source to the implant rechargeable battery ensures a power supply of the rechargeable battery by the external source and hence maintains, during the whole transportation and storage duration before implantation, a battery charge level higher than a minimum predetermined level. A protection support wedges the implant with respect to the accessory while ensuring the electrical coupling of the implant to the external source, thanks to a shock-absorbing structure and vibration-filtering structure, with a texture of elastically deformable strands or slats, wrapping and wedging the implant in position inside the protection support.

### BACKGROUND OF THE INVENTION

### Technical Field

The invention relates to medical devices during their transportation and storage, in the period between their manufacturing and the moment where they are used by the practitioner to be implanted in a patient.

It more particularly relates to those devices which incorporate a self-powering system comprising a mechanical energy harvesting device, also called “harvester” or “scavenger”, associated with an integrated energy storage component, such as a rechargeable buffer micro-battery or a high-performance capacitor.

The harvesting device can in particular be of the so-called “PEH” (Piezoelectric Energy Harvester) type, which uses as a mechanical-electrical transducer an oscillating piezoelectric beam coupled to an inertial mobile mass.

Transportation and storage of such devices involve, as will be explained hereinafter, a certain number of specific constraints and difficulties, related to the presence of the harvester, and that are not met with the conventional medical devices powered by a very long-life static battery (devices such as pacemakers, implantable defibrillators, etc.).

### State of the Art

The “harvester” devices are used in particular to power autonomous implantable medical devices (hereinafter “implants”), in particular autonomous capsules designed to be implanted into a heart cavity.

The invention is nevertheless not limited to such a device, it is also applicable to many other types of miniaturized implantable medical devices, whatever the operational purpose thereof, cardiac or other.

One of the critical aspects of these miniaturized devices is the power autonomy. Indeed, life duration of such a device being about 8 to 10 years, taking into account the very small sizes, it is not possible to use a conventional battery, even a high-density one. A harvester addresses this drawback by collecting the mechanical energy resulting from the various movements undergone by the implant body. These movements may have for origin a certain number of phenomena occurring in particular at the rhythm of the heartbeats, such as periodic shakes of the wall on which the implant is anchored, heart tissue vibrations linked i.a. to closings and openings of the heart valves, or also blood flow rate variations in the surrounding environment, which stress the implant and make it oscillate at the rhythm of the flow rate variations. The mechanical energy that is collected is converted through a suitable mechanical-electrical transducer into an electrical energy (voltage or current) sufficient for powering the various circuits and sensors of the device and charging the energy storage component. This power system allows the device to operate in full power autonomy for its whole lifetime.

This energy harvesting technique is particularly well adapted for powering the implanted autonomous capsules having no physical connection with a remote device. Such capsules are called for this reason “leadless capsules”, for distinguishing them from the electrodes or sensors arranged at the distal end of a lead, through the whole length of which run one or several conductors connected to a generator itself connected to the opposite, proximal end.

In this cardiac application case, the leadless capsule continuously monitors the patient's rhythm and if necessary issues to the heart electrical pulses for pacing, resynchronization and/or defibrillation in case of rhythm disorders detected by the capsule. The leadless capsule may be an epicardial capsule, fixed to the outer wall of the heart, or an endocavitary capsule, fixed to the inner wall of a ventricular or atrial cavity, or a capsule fixed to the wall of a vessel near the myocardium. The fixation of the capsule to the implantation site is made through a protruding anchoring system extending the capsule body and designed to penetrate the cardiac tissue, in particular by means of a screw.

The capsule comprises various electronic circuits, sensors, etc., as well as wireless communication transmission/reception means for the remote exchange of data, the whole being integrated in a body of very small size that can be implanted at sites whose access is difficult or that leave little space available, such as the apex of the ventricle, the inner wall of the atrium, etc. US 2009/0171408 A1 (Solem), US 2017/0151429 A1 (Regnier) and WO 2018/122244 A1 (Regnier) describe various examples of such intracardiac leadless capsules.

The energy harvesting device integrated to these capsules can in particular implement an inertial pendular unit subjected to the above-described external stresses: a mobile mass (called “seismic mass”) coupled to an elastically deformable element is driven according to the movements of the capsule and vibrates at a natural free oscillation frequency. The mechanical energy of the oscillation is converted into electrical energy by a mechanical-electrical transducer, in particular by a component such as a piezoelectric beam clamped at one of its ends and coupled to the inertial mass at its other end, which is free. The beam, that is cyclically and alternately stressed in bending, generates electrical charges that are collected at the surface of the component to be used by the self-powering system of the leadless capsule for powering the various circuits and sensors of the capsule and for charging the energy storage component.

Such an energy harvesting device of the PEH type for powering an implant from the oscillations of a piezoelectric beam is described in particular in U.S. Pat. No. 3,456,134 A (Ko) and in WO 2019/001829 A1 (Cairdac).

In the pre-implantation phase, i.e. during transportation and storage of the implant, several difficulties may appear.

A first problem lies in the necessity to maintain during the whole storage duration (which may last several months) a sufficient level of charge of the integrated buffer micro-battery to maintain the electric circuits of the implant in standby state until the moment of implantation, moment at which these circuits will be activated to become fully functional.

Of course, the consumption of the implant in standby state is very low, of the order of 1 μA, including the power supply of the standby circuits and the self-discharge current of the energy storage component. But the energy storage components used with the miniaturized implants, whether they are rechargeable buffer micro-batteries or high-capacity capacitors (hereinafter, the generic term “battery” will be used), have a limited capacity, typically with a value of the order of 1 mAh, which ensures a shelf life of about 1000 hours, i.e. approximately 40 days only—indeed, on shelf, the implant is stationary and there is therefore no charge by the harvester.

These values are to be compared to the traditional implants' ones (pacemakers, implantable defibrillator, etc.) provided with a long-life static battery, whose capacity is generally of at least 100 mAh: in this case, for a same current of 1 μA, after 20 months of storage the battery will have lost only 15% of its nominal capacity.

For such conventional implants, WO 98/08567 A1 (Pacesetter) proposes to couple to the implant an external source of energy for powering the implant during the whole storage duration, thus preserving the internal power battery of the latter. An interface circuit makes it possible to switch at will the power supply of the implant circuits, either to the external source or to the internal battery.

U.S. Pat. No. 6,181,105 B1 (Cutolo et al.), US 2008/103557 A1 (Davis et al.), US 2003/204218 A1 (Vogel et al.) and US 2005/075695 A1 (Schommer) disclose other systems for powering or charging an implantable medical device from an external source of energy.

A second problem, specific to the implants provided with a harvester, is the fragility of the mechanical-electrical transducer and the risks of damaging the latter during the transportation—a problem that of course does not exist with the implants provided with a static battery.

In particular, in the case of a harvester with a piezoelectric-beam pendular unit, this assembly risks to be subjected, during the transportation, to far higher accelerations than those for which it has been designed to operate after implantation in situ. It is also liable to undergo vibrations at frequencies close to the free oscillation frequency of the mass-spring assembly of the pendular unit, with, in this case, a risk of resonance generating excessive amplitudes and stresses, deleterious for the integrity of the piezoelectric beam.

Another object of the invention is to protect the implant against this risk of damaging the harvester during the transportation between the manufacturing plant and the final storage place.

US 2019/070422 A1 (Regnier) describes a leadless capsule before implantation, configured for its storage and transportation.

The problem considered by this document is however different: the matter is to ensure in this situation an exchange of data between the capsule and the external environment by an intracorporeal communication technique called HBC (Human Body Communication), which is a technique in which the communication is conducted through a medium consisting of the body tissues or interstitial fluids of a patient and can therefore normally only be implemented after implantation. But the capsule powering problem is not contemplated, as the capsule is supposed to be in a functional state.

### SUMMARY OF THE INVENTION

To solve the above-mentioned problems, the invention proposes an accessory for transportation and storage of an implant comprising, in a manner known per se, an external source of electrical energy for the temporary storage of an electrical energy during the transportation and storage of the implant, the external source being physically separated from the implant, and a temporary electrical coupling link from the external source to the implant.

The implant is an implant comprising a tubular body housing an energy harvesting module adapted to convert external stresses applied to the implant into electrical energy, by means of an inertial pendular unit comprising an elastically deformable element coupled to an inertial mass, and a rechargeable battery adapted to be recharged by the energy harvesting module, the battery being previously charged at an initial charge level.

The electrical coupling link is a link to the implant rechargeable battery, in such a way as to ensure a power supply of the rechargeable battery by the external source and hence to maintain, during the whole transportation and storage duration before implantation, a battery charge level higher than a minimum predetermined level.

The accessory further comprises a protection support for receiving and wedging the implant with respect to the accessory in a configuration ensuring the electrical coupling of the implant to the external source. This protection support comprises a shock-absorbing and vibration-filtering structure, with a texture of elastically deformable strands or slats, wrapping and wedging the implant in position inside the protection support.

Advantageously, the protection support comprises electrical terminals connected to the external source and adapted to come into contact with respective surface electrodes of the implant tubular body, the surface electrodes being coupled, inside the implant, to the rechargeable battery. These electrical terminals can in particular comprise retractable touch tips protruding inside the protection support and adapted to come into radial contact against the respective surface electrodes of the implant tubular body.

The elastically deformable strands or slats of the protection support are advantageously adapted, in deformed configuration, in contact with the implant, to come into tangential contact with the surface of the implant tubular body. Preferably, the strand or slat texture is laterally permeable to gas, in such a way as to allow, in a configuration in which the implant is wrapped and wedged in the absorbing structure, the circulation up to the implant of a sterilization gas introduced from the outside of the absorbing structure.

The protection support can also comprise an elastically deformable axial stop adapted to come into contact with a front and/or back end of the implant tubular body.

Advantageously, the accessory further comprises a component for limiting the current delivered by the external source to the implant rechargeable battery through the temporary electrical coupling link, and/or a control indicator for the coupling and/or the passage of a current from the external source to the implant rechargeable battery through the temporary electrical coupling link, and/or a circuit for evaluating the battery charge level, and a circuit adapted to interrupt the power supply of the rechargeable battery by the external source when the evaluated charge level exceeds a predefined high threshold, and to reestablish the power supply of the rechargeable battery by the external source when the evaluated charge level reaches a predefined low threshold.

The invention has also for object a packaging for the transportation and storage of an implant as defined hereinabove, this packaging comprising a sealed package defining a sterile internal volume enclosing the implant and comprising an accessory as defined hereinabove.

The external source can be a non-rechargeable electric cell housed inside the sealed package, or an inductive energy receiver housed inside the sealed package and non-galvanically coupled to an inductive energy emitter external to the sealed package.

### BRIEF DESCRIPTION OF THE DRAWINGS

FIG. 1 illustrates a leadless capsule in its environment, implanted in the bottom of the right ventricle of a patient's myocardium.

FIG. 2 is a longitudinal general view of a leadless capsule comprising a pendular unit based energy harvester.

FIG. 3 schematically shows the main functional blocks constituting a leadless capsule.

FIG. 4 is a general view illustrating the full packaging, with the implant and its accessories enclosed in a sterile sealed package.

FIG. 5 illustrates more precisely the support for protection and wedging of the implant, in situation inside the sealed package, during the transportation and the storage.

FIG. 6 is a variant of FIG. 5, in which the “stick” cell has been replaced by a “button” cell.

FIG. 7 illustrates, in perspective view, the implant placed inside one of the two deformable parts constituting the protection and wedging support.

FIG. 8 is an elevation view, in partial cross-section, corresponding to FIG. 7.

FIG. 9 is an end view corresponding to FIG. 7.

FIG. 10 is homologous to FIG. 7, only with the single deformable part and without the implant.

FIG. 11 is a top view of the deformable part illustrated in FIG. 10.

FIG. 12 is a detail view, in elevation, of the front part of the implant in rest against a front stop of the protection support.

FIG. 13 is homologous to FIG. 12, in cross-sectional view.

FIG. 14 is an electrical diagram explaining how the system for recharging the implant buffer battery operates.

### DETAILED DESCRIPTION OF PREFERENTIAL EMBODIMENTS OF THE INVENTION

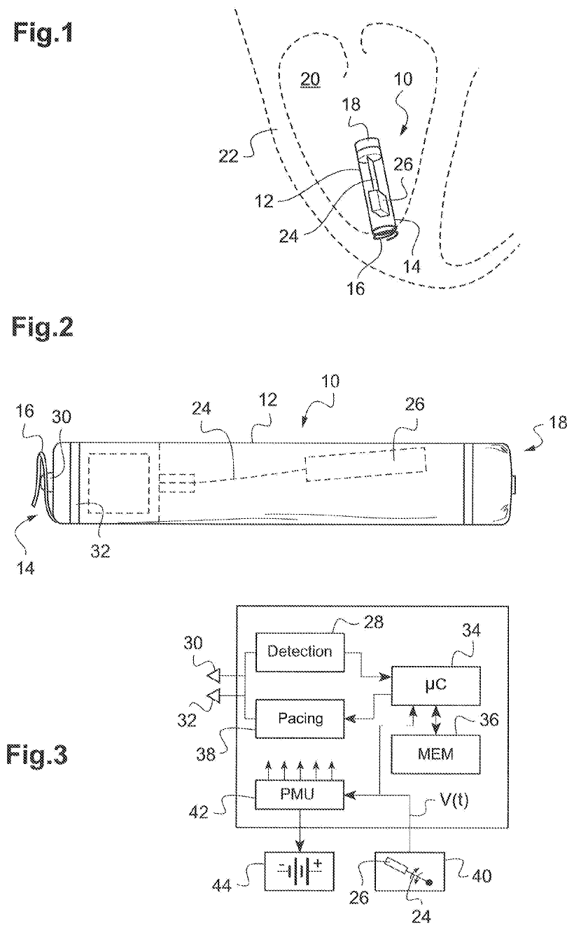

In FIGS. 1 and 2 is shown an implant of the leadless capsule type 10 in an application to cardiac pacing.

Capsule 10 has the external form of an elongated cylindrical tubular body 12 enclosing the various electronic and power circuits of the capsule, as well as a pendular unit based energy harvester. The typical size of such a capsule is about 6 mm diameter for about 25 to 40 mm length.

Tubular body 12 has, at its front (distal) end 14, a protruding anchoring element, for example an helical screw 16, to hold the capsule on the implantation site. The opposite (proximal) end 18 of capsule 10 is a free end, which is only provided with means for the temporary connection to a guide-catheter (not shown) or another implantation accessory used for implantation or explantation of the capsule.

In the example illustrated in FIG. 1, the leadless capsule 10 is an endocavitary implant implanted into a cavity 20 of myocardium 22, for example at the apex of the right ventricle. As an alternative, still in an application to cardiac pacing, the capsule can also be implanted on the interventricular septum or on an atrial wall, or also be an epicardial capsule placed on an external region of the myocardium. Leadless capsule 10 is moreover provided with an energy harvesting module comprising an inertial pendular unit that oscillates, inside the capsule, following the various external stresses to which the capsule is subjected. These stresses may result in particular from: movements of the wall to which the capsule is anchored, which are transmitted to tubular body 12 by anchoring screw 16; and/or blood flow rate variations in the environment surrounding the capsule, which produce oscillations of tubular body 12 at the rhythm of the heartbeats; and/or various vibrations transmitted by the heart tissues. The pendular unit can in particular be consisted of a piezoelectric beam 24 clamped at one of its ends, and whose opposite, free end is coupled to a mobile inertial mass 26, the whole forming a pendular system of the mass-spring type. Due to its inertia, mass 26 subjects beam 24 to a deformation of the vibratory type on either side of a neutral or non-deformed position corresponding to a stable rest position in the absence of any stress. Piezoelectric beam 24 further performs a mechanical-electrical transducer function for converting the mechanical bending stress that is applied to it into electric charges that are collected to produce an electrical signal that, after rectification, stabilization and filtering, will power the various electronic circuits of the capsule.

FIG. 3 is a synoptic view of the various electric and electronic circuits integrated to the leadless capsule, presented as functional blocks.

Block 28 denotes a heart depolarization wave detection circuit, which is connected to a cathode electrode 30 in contact with the heart tissue and to an associated anode electrode 32, for example a ring electrode formed on the tubular body of the capsule (see FIG. 2). Detection block 28 comprises filters and means for analog and/or digital processing of the collected signal. The so-processed signal is applied to the input of a microcomputer 34 associated with a memory 36. The electronic unit also includes a pacing circuit 38 operating under the control of microcomputer 34 to issue, as needed, myocardial pacing pulses to the system of electrodes 30, 32.

An energy harvesting circuit PEH 40 is moreover provided, consisted by the pendular unit formed by piezoelectric beam 24 and inertial mass 30, described hereinabove with reference to FIGS. 1 and 2. Piezoelectric beam 24 also ensures a mechanical-electrical transducer function that converts into electrical charges the mechanical stresses undergone and produces a variable electrical signal V(t), which is an alternating signal oscillating at the natural oscillation frequency of the pendular beam 24/mass 30 unit, and at the rhythm of the successive beats of the myocardium to which the capsule is coupled.

The variable electrical signal V(t) is sent to a power management unit or PMU 42. PMU 42 rectifies and regulates the signal V(t) so as to output a stabilized direct voltage or current used to power the various electronic circuits and to charge an integrated buffer micro-battery 44 (to the case of a micro-battery will be equated that of a high-capacity capacitor, which fulfils the same function of temporary storage of an electrical energy for ensuring the power supply of all the circuits of the implant).

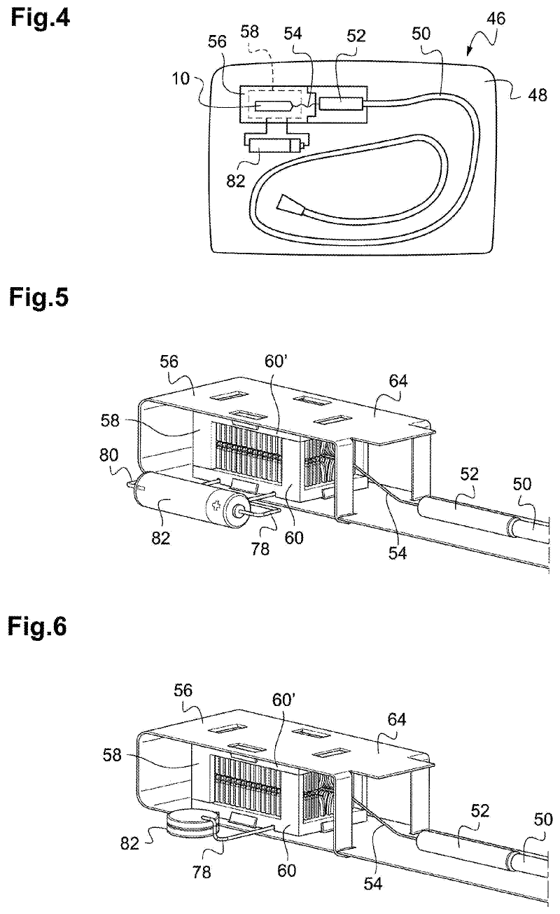

FIG. 4 is a general view illustrating the full packaging, with the implant and its accessories enclosed in a sterile sealed package.

The packaging comprises, with a sealed package 46 defining a sealed and sterile internal volume 48, in which capsule 10 is enclosed. The package also contains, in addition to the capsule, a catheter 50 the implantation, which is ended, on the distal end (near the capsule), by a “housing” 52 receiving and protecting the capsule during the guiding into the venous network and also preventing anchoring screw 16 to injure the vessel walls. In the package, the capsule is out of housing 52 and is connected to the catheter only by a security thread or “Ariane's thread” 54, from which it will be disconnected only once the definitive implantation reached.

Capsule 10 is arranged inside a protection and wedging support 56, including an absorbing structure 58 characteristic of the invention.

FIGS. 5 to 13 illustrate in more detail the way this protective support 56 is made.

Support 56 comprises an absorbing structure 58 inside which capsule 10 is housed, in such a way as to protect the latter from the shocks and to filter the potential vibrations it could undergo, in particular at frequencies close to the free resonant frequency of the harvester pendular unit, in such a way as not to risk damaging the latter by excessive deformation amplitudes or shocks during the transportation.

Absorbing structure 58 is consisted of two identical deformable parts 60, 60′, mounted against each other, in such a way as to sandwich capsule 10 and to wedge the latter in position.

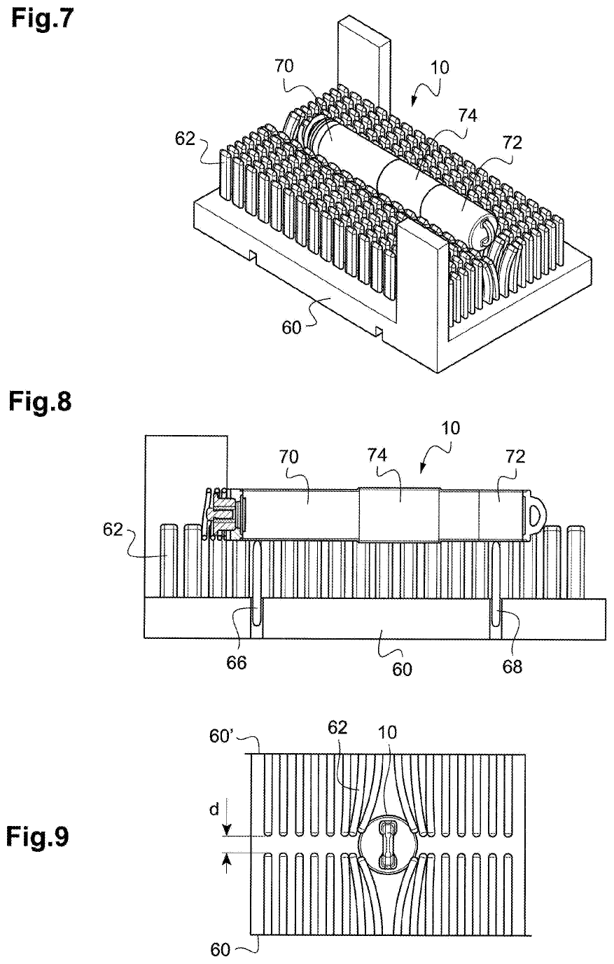

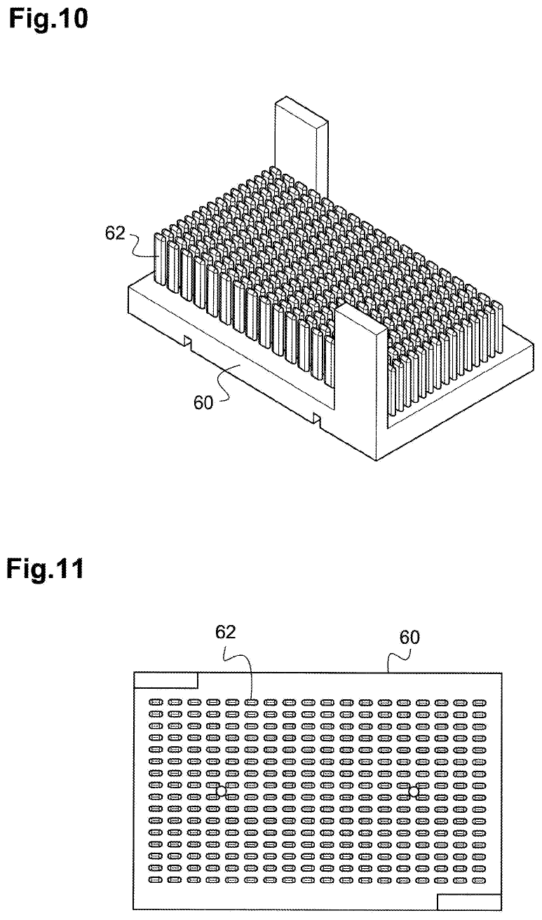

FIGS. 7 to 9 illustrate in perspective, elevation and end view, respectively, one of these two deformable parts 60 with capsule 10 placed at the center, whereas FIGS. 10, 11 illustrate in isolation, in perspective and top view, respectively, the only deformable part 60.

Part 60 is in the form of a flat base supporting a strand or slat texture 62, for example an assembly made single-piece by molding or injection of a flexible material such as polyethylene, polypropylene, nylon, silicon or thermoplastic polyurethane, preferably with a rigidity lower than 80 Shore A. The strands or slats 62 may be in the form of wires, fibers or slats; in this latter case, as illustrated in the figures, the greatest size of each slate is preferably parallel to the main axis of the capsule, in such a way as to favor the bending deformation: as can be seen in particular in FIG. 9, the strands or slats enter into tangential contact with the surface of capsule 10, and the tangential pressure exerted on the latter can potentially be adjusted by adapting the spacing d between the two opposite deformable parts 60, 60′.

As an alternative, the flexible strands or slats 62, deformable in bending, may be replaced by a massive block of foam. However, the use of a strand or slat texture makes it possible to provide between these strands or slats a space that makes the texture laterally permeable to gas: it is hence possible to make circulate up to the capsule a sterilization gas introduced from the outside of absorbing structure 58, even in a configuration in which the capsule is still wrapped and wedged in the absorbing structure.

The strands or slats elasticity, size and number are moreover advantageously chosen in such a way as to provide against the vibrations a filtering that is maximum for frequencies lower than about 40 Hz, i.e. in the field of vibrations the most liable to damage piezoelectric beam 24 by resonance of the inertial pendular unit.

Thanks to absorbing structure 58, the assembly is hence protected against shocks and vibrations in the three dimensions X, Y and Z.

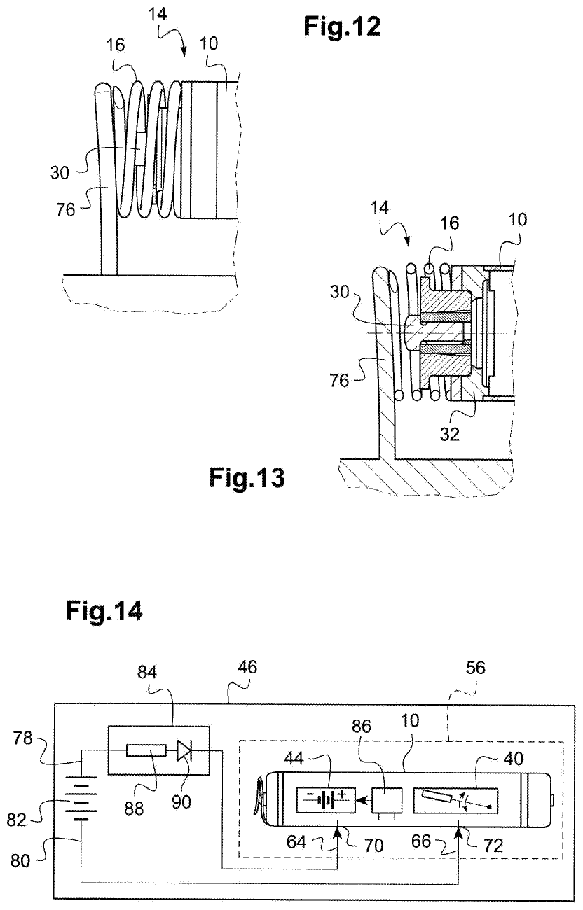

As illustrated in FIGS. 12 and 13, deformable part 60 advantageously comprises an axially deformable front stop 76 for protecting in particular the distal pacing electrode 30 in such a way that no element can enter into contact with the latter. Indeed, this type of electrode is usually covered with a coating of the titanium nitride type of very small thickness (less than 10 μm), very efficient as regards the pacing but very fragile and sensitive to scratches and particulate contaminations. The axial stiffness of anchoring screw 16 is moreover adapted to be higher than the force applied by one or several axial stops 76, typically higher than 1 N.

Referring again to FIG. 5, absorbing structure 58, formed of the two deformable parts 60, 60′ enclosing capsule 10, is housed in a rigid part 64, for example a part cut from a material such as polypropylene, polyethylene, or other, then folded with formation of stops for holding absorbing structure 58 in position. The rigid part 64 also supports the end of catheter 50 and the housing 52 near the capsule enclosed in absorbing structure 58.

In addition to the mechanical protection, it is provided to establish an electrical coupling with electrodes of the capsule, in such a way as to be able to recharge as needed the buffer battery integrated to the capsule, as will be explained hereinafter with reference to FIG. 14.

For that purpose, touch tips 66, 68 (FIG. 8) are provided, coming into contact with distinct conductive surfaces 70, 72 of the tubular body 12 of capsule 10.

Such a tubular body structure comprising two conductive (metallic) surfaces 70, 72, separated by an isolating (ceramic) cylindrical surface 74, is described for example in US 2020/338241 A1 (Regnier et al.), hereby incorporated by reference.

Touch tips 66, 68 may be rods with a telescopic end or a retractable ball coming into contact with conductive surfaces 70, 72; as an alternative, the electrical coupling may be made through flexible blades or conductive springs, or through any other means fulfilling the same function.

As illustrated in particular in FIG. 5, touch points 66, 68 are connected by respective conductors 78, 80 to a source of electrical energy 82, offset with respect to the capsule protection and wedging support 56.

The source of electrical energy can be in particular a conventional “stick” cell of 1.5 V (as illustrated in FIG. 5) or a “button” battery of smaller size (as in the alternative illustrated in FIG. 6), according to the desired energy capacity, essentially linked to the guaranteed storage duration, as will be explained hereinafter. As an alternative, the offset source of electrical energy can be an inductive energy receiver, for example an inductive charging loop placed in the internal volume 48 of the sterile packaging of package 46; this loop is then coupled to an inductive energy emitter located outside the sterile packaging 46. The way the charge level of the buffer battery 44 can be maintained at a satisfying minimum level despite the absence of charge by harvester 40 will now be described with reference to the electrical diagram of FIG. 14.

Conductors 78, 80 and touch points 66, 68 ensure a galvanic coupling between capsule 10 and the offset source of electrical energy 82 (hereinafter called “cell” for the sake of simplicity).

The nominal voltage of cell 82 is chosen in such a way as to be higher than the operational voltage of buffer battery 44, for example a cell voltage of 1.5 to 9 C, typically of 5 to 6 V, for a buffer battery voltage typically varying between 3 V and 4.2 V. If the cell voltage is lower than that of the buffer battery, a voltage booster circuit can be provided, either external to the capsule, or internal to the latter (for example, a voltage boost stage within PMU 42 (FIG. 3)).

The coupling of cell 82 to buffer battery 44 comprises, in addition to conductors 78, 80 and touch points 64, 66, an interface circuit 84 between cell 82 and capsule 10, and an interface circuit 86 internal to the capsule for coupling the external conductive surfaces 70, 72 to buffer battery 44.

In its simplest configuration, the battery/capsule interface circuit 84 comprises a resistance 88 for limiting the charging current delivered by the cell, and a diode 90 for interrupting the charging when the voltage level of battery 44 reaches the voltage value of cell 82. In a more elaborate alternative, the interface circuit 84 can comprise a circuit for determining the voltage level of the battery and controlling selectively the delivery of the charging current, by interrupting the power supply of battery 44 by cell 82 when the charge level exceeds a predefined high threshold, and by reestablishing this power supply when the charge level falls down to a predefined low threshold.

Moreover, a means can be provided, for example a LED (not shown), for visually controlling the correct coupling between cell 82 and capsule 10, i.e. for checking the good condition of the function of controlled charging of the capsule buffer battery by cell 82 inside the sealed packaging.

From a quantitative point of view, for a standby current and a self-discharge of the battery producing a permanent current of the order of 1 μA and for a capacity of the battery of the order of 1 mAh, a shelf life of about 1000 h, i.e. about 40 days, is normally obtained, due to the absence of charge by the harvester, which is immobile.

To guarantee a storage duration of 24 months during which the capsule must remain functional although being in standby state, it is necessary to provide for about 30 charge cycles of battery 44 by cell 82. With an estimated operation efficiency of 50%, the external cell 82 has a capacity of 60 mAh, a value fully compatible with that provided by the conventional “button” cells, which have typically a capacity of the order of 80 to 100 mAh or more.

The invention hence makes it possible to guarantee, with very simple means, in any circumstances, a very long term shelf storage, without any reduction of longevity of the capsule, the latter being always functional and ready to be awake at any time for its implantation.

1. An accessory for transportation and storage of an autonomous cardiac implant of the leadless capsule type before implantation,
the accessory comprising an external source of electrical energy for the temporary storage of an electrical energy during the transportation and storage of the implant, the external source being physically separated from the implant, and a temporary electrical coupling link from the external source to the implant,
wherein the implant comprises a tubular body which houses:
an energy harvesting module adapted to convert external stresses applied to the implant into electrical energy, by means of an inertial pendular unit comprising an elastically deformable element coupled to an inertial mass; and
a rechargeable battery adapted to be recharged by the energy harvesting module, the battery being previously charged at an initial charge level,
wherein the electrical coupling link is a link to the implant rechargeable battery, in such a way as to ensure a power supply of the rechargeable battery by the external source and hence to maintain, during the whole transportation and storage duration before implantation, a battery charge level higher than a minimum predetermined level; and
wherein the accessory further comprises a protection support for receiving and wedging the implant with respect to the accessory in a configuration ensuring the electrical coupling of the implant to the external source, wherein the protection support comprises a shock-absorbing and vibration-filtering structure,
wherein said shock-absorbing and vibration-filtering structure comprises a texture of elastically deformable strands or slats, wrapping and wedging the implant in position inside the protection support.

2. The accessory according to claim 1, wherein the protection support further comprises electrical terminals connected to the external source and adapted to come into contact with respective surface electrodes of the implant tubular body, the surface electrodes being coupled, inside of the implant, to the rechargeable battery.

3. The accessory according to claim 2, wherein the electrical terminals comprise retractable touch tips protruding inside the protection support and adapted to come into radial contact against the respective surface electrodes of the implant tubular body.

4. The accessory according to claim 1, wherein the elastically deformable strands or slats are adapted, in a deformed configuration in contact with the implant, to come into tangential contact with the surface of the implant tubular body.

5. The accessory according to claim 1, wherein the strand or slat texture is laterally permeable to gas, in such a way as to allow, in a configuration in which the implant is wrapped and wedged in the absorbing structure, a circulation up to the implant of a sterilization gas introduced from the outside of the absorbing structure.

6. The accessory according to claim 1, wherein the protection support further comprises an elastically deformable axial stop adapted to come into contact with a front and/or back end of the implant tubular body.

7. The accessory according to claim 1, further comprising a component for limiting the current delivered by the external source to the implant rechargeable battery through the temporary electrical coupling link.

8. The accessory according to claim 1, further comprising a control indicator for the coupling and/or the passage of a current from the external source to the implant rechargeable battery through the temporary electrical coupling link.

9. The accessory according to claim 1, further comprising a circuit for evaluating the battery charge level, and a circuit adapted to interrupt the power supply of the rechargeable battery by the external source when an evaluated charge level exceeds a predefined high threshold, and to reestablish the power supply of the rechargeable battery by the external source when the evaluated charge level reaches a predefined low threshold.

10. A packaging for the transportation and storage of an autonomous cardiac implant of the leadless capsule type before implantation, comprising:
a sealed package defining a sterile internal volume enclosing the implant, wherein the implant comprises a tubular body which houses:
an energy harvesting module adapted to convert external stresses applied to the implant into electrical energy, by means of an inertial pendular unit comprising an elastically deformable element coupled to an inertial mass, and
a rechargeable battery adapted to be recharged by the energy harvesting module, the battery being previously charged at an initial charge level,
wherein the packaging further comprises, inside the sterile volume, an accessory comprising:
an external source of energy for the temporary storage of an electrical energy during the transportation and storage of the implant, the external source being physically separated from the implant;
a temporary electrical coupling link from the external source to the implant, in such a way as to ensure a power supply of the rechargeable battery by the external source and hence to maintain, during the whole transportation and storage duration before implantation, a battery charge level higher than a minimum predetermined level; and
a protection support adapted to receive and wedge the implant with respect to the accessory in a configuration ensuring the electrical coupling of the implant to the external source, wherein the protection support comprises a shock-absorbing and vibration-filtering structure,
wherein the shock-absorbing and vibration-filtering structure comprises a texture of elastically deformable strands or slats, wrapping and wedging the implant in position inside the protection support.

11. The packaging of claim 10, wherein the external source is a non-rechargeable electric cell housed inside the sealed package.

12. The package of claim 10, wherein the external source is an inductive energy receiver housed inside the sealed package and non-galvanically coupled to an inductive energy emitter external to the sealed package.
